(19) United States
(12) Patent Application Publication
(10) Pub. No.: US 2010/0268263 A1
(43) Pub. Date: Oct. 21, 2010

(54) EMBOLIC PROTECTION FILTERS, FILTER MEMBRANES, AND METHODS FOR MAKING AND USING THE SAME

(75) Inventors: Peter Edelman, Maple Grove, MN (US); Nayan Ashar, San Jose, CA (US); Narin Anderson, Savage, MN (US); Bradley White, Palo Alto, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/426,988

(22) Filed: Apr. 21, 2009

(51) Int. Cl. A61B 17/22; B23P 11/00; B23P 19/04

(52) U.S. Cl. 606/200; 29/428; 29/460

(57) ABSTRACT

Embolic protection filters, filter membranes, and methods for making and using the same. An example embolic protection filter may include an elongate shaft having a proximal region and a distal region. A filter frame may be to the distal region. A filter membrane may be attached to the filter frame. The filter membrane may have a plurality of apertures formed therein. The filter membrane may include a polymer having a bulk portion and a surface portion. The surface portion may include a surface modification.

### FIELD OF THE INVENTION

The present invention pertains to embolic protection filters, filter membranes, and methods for making and using embolic protection filters and filter membranes. More particularly, the present invention pertains to filter membranes that include a polymer having a bulk portion and a surface portion.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of filtering devices have been developed for medical use, for example, intravascular use. Of the known filtering devices, each has certain advantages and disadvantages. There is an ongoing need to provide alternative filtering devices as well as alternative methods for manufacturing filtering devices.

### BRIEF SUMMARY

The disclosure pertains to design, material, manufacturing method, and use alternatives for embolic protection filters, filter membranes, and the like. An example embolic protection filter may include an elongate shaft having a proximal region and a distal region. A filter frame may be to the distal region. A filter membrane may be attached to the filter frame. The filter membrane may have a plurality of apertures formed therein. The filter membrane may include a polymer having a bulk portion and a surface portion. The surface portion may include a surface modification.

Another example embolic protection filter may include an elongate shaft having a proximal region and a distal region. A filter frame may be attached to the distal region. A filter membrane may be attached to the filter frame. The filter membrane may include a spider silk or other naturally occurring super strong polymer. The filter membrane can also contain a liquid crystalline polymer (LCP) such as Kevlar® or Vectran® or other known LCPs.

An example method for manufacturing a filter membrane for an embolic protection filter may include providing an elongate shaft having a proximal region and a distal region, attaching a filter frame to the distal region, and attaching a filter membrane to the filter frame. The filter membrane may have a plurality of apertures formed therein. The filter membrane may include a polymer having a bulk portion and a surface portion. The surface portion may include a surface modification.

Another example method for manufacturing an embolic protection filter may include providing a filter frame, providing a mandrel, disposing the filter frame on the mandrel, and spray coating a filter membrane onto the mandrel and the filter frame. The filter membrane may include a polymer having a bulk portion and a surface portion. The surface portion may include a surface modification.

Another example embolic protection filter may include an elongate shaft having a proximal region and a distal region. A filter frame may be attached to the distal region. A filter membrane may be attached to the filter frame. The filter membrane may include a polymer containing a low surface energy segment, such as a fluorinated segment, a siloxane segment or a hydrocarbon segment. These segments can either be located in the middle of the polymer backbone or preferentially at the ends of the polymer chains.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

FIG. 1 is a partial cross-sectional side view of an example embolic protection filter disposed in a blood vessel; and

FIG. 2 is a side view depicting an example mandrel used in the manufacturing of an example embolic protection filtering device.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

When a clinician performs an intravascular intervention such as angioplasty, atherectomy, and the like, embolic debris may dislodge from the blood vessel that can travel in the bloodstream to a position where it may impair blood flow, possibly leading to tissue damage. A number of other situations and/or interventions may also result in the mobilization of embolic debris. Accordingly, embolic protection filtering devices have been developed that can be disposed in the blood vessel downstream of the treatment site and expanded to capture debris.

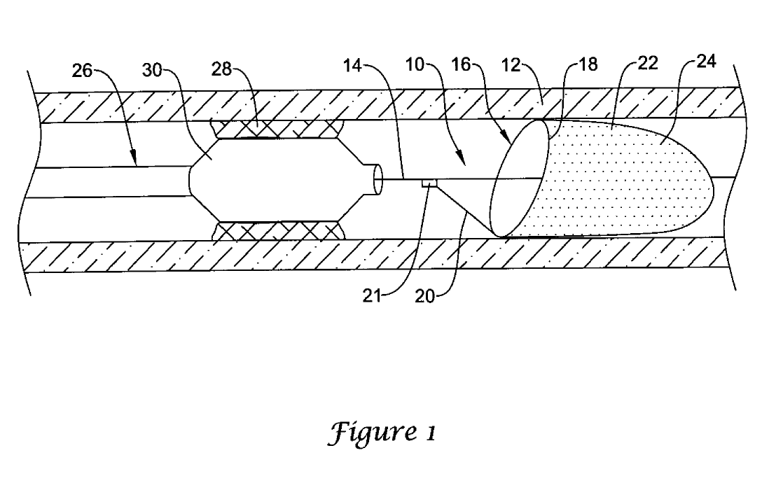

FIG. 1 is a partial cross-sectional view of an example embolic protection filtering device 10 disposed within a blood vessel 12. Device 10 may include an elongate shaft or filter wire 14 having an embolic protection filter 16 coupled thereto. Filter 16 includes a filter frame or loop 18 and a filter membrane or fabric 22 coupled to filter loop 18. Filter membrane 22 can be drilled (for example, formed by known laser techniques) or otherwise manufactured to include a plurality of apertures 24. These holes or apertures 24 can be sized to allow blood flow therethrough but restrict flow of debris or emboli floating in the body lumen or cavity.

In general, filter 16 may be adapted to operate between a first generally collapsed configuration and a second generally expanded configuration for collecting debris in a body lumen. To this end, in at least some embodiments, loop 18 may be comprised of a “self-expanding” shape-memory material such as nickel-titanium alloy, which is capable of biasing filter 16 toward being in the second expanded configuration. Additionally, filter loop 18 may include a radiopaque material or include, for example, a radiopaque wire disposed about a portion thereof. Some further details regarding these and other suitable materials are provided below.

One or more struts 20 may extend between filter loop 18 and filter wire 14. Strut 20 may be coupled to filter wire 14 by a coupling 21. Coupling 21 may be one or more windings of strut 20 about filter wire 14 or may be a fitting disposed over an end of strut 20 to attach it to filter wire 14. The exact arrangement of struts 20 can vary considerably. One of ordinary skill in the art would be familiar with the various arrangements of struts 20 that are appropriate for a given intervention.

With filter 16 properly positioned in blood vessel 12, another medical device may be advanced over filter wire 14 in order to treat and/or diagnose a lesion 28. For example, a catheter 26 (such as the balloon catheter depicted in FIG. 1) may be advanced over filter wire 14 in order to expand lesion 28. Of course numerous other devices could just as easily be passed over filter wire 14 including any device designed to pass through an opening or body lumen. For example, the device may comprise any type of catheter (e.g., therapeutic, diagnostic, or guide catheter), a stent delivery catheter, an endoscopic device, a laproscopic device, variations and refinements thereof and the like, or any other suitable device. Alternatively, another device may be advanced over or through its own guiding structure to a suitable location adjacent filter 16 in a manner that allows device 10 to perform its intended filtering function.

Filtering device 10 is generally designed to filter embolic debris that might be generated during the course of this medical intervention. For example, device 10 can be used to capture embolic debris that might be generated during the use of catheter 26 such as when a balloon 30 (coupled to catheter 26) is inflated. It should be noted, however, that device 10 may find utility in concert with essentially any procedure that has the potential to loosen and release embolic debris in to the blood stream or with the devices associated with such procedures.

During a filtering procedure, blood will tend to flow through openings 24 in membrane 22. If membrane 22 is made from a thrombogenic material, clotting and/or emboli formation may occur. This may block or clog openings 24, which may undermine the purpose of filtering device 10. Because of this, it may be desirable for membrane 24 to be formed from a substantially non-thrombogenic material and/or a material with a non-thrombogenic or non-coagulant surface.

Some of the materials that may be used to form membrane 22 may include polymers with modified end groups. These polymers may also be termed polymers with surface modifying end groups (SMEs). Polymers with SMEs may, in general, include a polymer having a main chain portion (bulk portion) and an end group portion (surface portion). Since the end group portion has a lower surface energy than the mid-chain portions, it is thermodynamically driven to migrate to the surface, thereby lowering the surface energy of the entire polymer layer or film. In some embodiments, mixed solvents having components with more than one evaporation rate may be used to modify the degree to which the surface composition differs from the bulk composition. The bulk portion may include any conventional elastic, strong, film forming material such as those materials that may be used to manufacture typical filter membranes.

The surface portion may also be achieved by a surface modification. These surface modifications may include plasma polymerization by plasma treatment or some process that reactively couples a surface desirable molecule to the surface of the polymer, e.g. a grafting reaction. For example, the surface portion may result in a modified surface on the bulk portion that may provide desirable properties to membrane 22 such as non-thrombogenic properties. In addition, the surface portion may also have enhanced lubricity (e.g., due to the hydrophilic and slippery nature of surface portion) that may enhance usability (less likely to catch on other components of filtering device 10, more likely to travel down the arterial pathway without causing trauma, more likely to track through the tortuous path narrow opening of a calcified lesion), trackability, manufacturability (easier to remove membrane 22 from a mandrel that may be utilized during manufacturing), etc. Forming membrane from a lubricious material would obviate the need to add a lubricious coating, thus saving time and material costs associated with such procedures.

One of the most significant benefits of a surface modification can be that the filter can be packaged in a very tightly constrained state, ready to deliver. The problem with most polymers suitable for a filter application is that they tend to stick to themselves after a period of time (tack, blockiness). This is especially true after sterilization (electron beam, gamma irradiation, ethylene oxide) and storage periods of preferably greater than 2 years. The filter needs to be very tightly wrapped to achieve a narrow profile (<3 Fr (1 mm)). The modified surface portion described herein can decrease the tendency for the polymer to stick to itself so that on deployment the filter is able to open to its full diameter to achieve optimum wall apposition. In some embodiments, the filter may include a modified surface layer on one major surface. In other embodiments, the filter may include a modified surface layer on both major surfaces.

In some embodiments, the bulk layer may include one or more polymers. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVDC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the polymer may include or can be blended with a liquid crystal polymer (LCP), for example, up to about 6% LCP.

The surface portion may be formed of the same or a different polymer than the bulk portion. Thus, the surface portion may include any of those materials listed above, as long as the bulk portion is different than the surface portion. In addition, as suggested above, it may be desirable for the surface formed from the surface migrating end group portions to have non-thrombogenic properties. Consequently, it may be desirable for the surface portion to be made from or include materials that have non-thrombogenic properties. Some examples of such materials may include biologically derived macromolecules such as heparin and hyaluronic acid. In addition, a number of synthetically derived non-thrombogenic materials may be used including polyethylene glycols of various molecular weights, pluronics (e.g., triblock copolymers of polypropylene oxide sandwiched between two end blocks of polyethylene oxide, commercially available from BASF), polyacrylamide and polyacrylamide copolymers, polyvinylpyrrolidone and polyvinylpyrrolidone copolymers, and the like, or any other suitable materials.

In forming membrane, the material(s) for bulk portion and surface portion may be mixed together. Because the material(s) utilized for surface portion may be chemically incompatible with the material(s) utilized for bulk portion, the surface portion materials may tend to phase separate and migrate to the surface or periphery of the polymer. This may desirably place the surface portion materials at the desired “surface” location and form the “surface modification” in the surface portion of membrane 22.

Alternatively, the bulk portion may comprise a layer of material and surface portion may comprise another layer of material that is applied to the bulk portion layer. For example, if the bulk portion is spray coated onto a mandrel, the surface portion can be spray coated or otherwise applied onto the bulk portion so as to form the desired membrane 22. The precise process and process steps would vary, of course, depending on the materials selected and the method of manufacturing. For example, it may be desirable for multiple base and/or surface portions to be utilized in a given membrane 22. In some embodiments, a surface agent may be added to the bulk portion. This may help base layer adhere to the surface portion and, consequently, the surface agent may function as a tie layer that helps to hold bulk portion together with surface portion.

In some other embodiments, filter membrane 22 may lack a surface portion or may simply be made from a singular or unitary polymeric materials. In these examples, the material selected may be chosen based on its superior chemical and environmental resistance as well as superior strength. Some of the materials that may be suitable for forming filter membrane 22 may include thermo-tropic liquid crystalline polymers, poly-ether-ether ketones, poly-ether-imide, and the like. These polymers may be dissolved in a suitable solvent (e.g., dimethyl formamide, tetrahydrofuran, N-methyl pyrrolidone, or the like). The dissolved polymer solutions may have a long, stable shelf life and may be used to manufacture membrane 22 in any suitable manner including spray forming (described in more detail below), dip coating, casting, and the like.

In other embodiments, filter membrane 22 may include a perfluoropolymers such as a perfluropolyether urethane. Perfluoropolymers are known to crosslink more readily than undergo chain scission on irradiation. This property may be utilized when forming membranes like membrane 22 that are to become crosslinked. Crosslinking may increase the strength of filter membrane 22. In addition, perfluoropolymer soft segment containing polyether urethanes can be formulated to achieve a spray formable solution of stable viscosity. In some embodiments, apertures 24 may be formed in filter membrane 22 prior to crosslinking. Alternatively, apertures 24 may be formed after crosslinking.

In still other embodiments, filter membrane 22 may include a spider silk or the like. These natural polymers have a desirably high strength to weight ratios. Membranes 22 may be formed by weaving spider silk fibers into a suitable net or sheet or configuration of material. The sheet may be woven to define apertures 24 or apertures 24 may be formed in the sheet. Nonwoven sheets may also be employed.

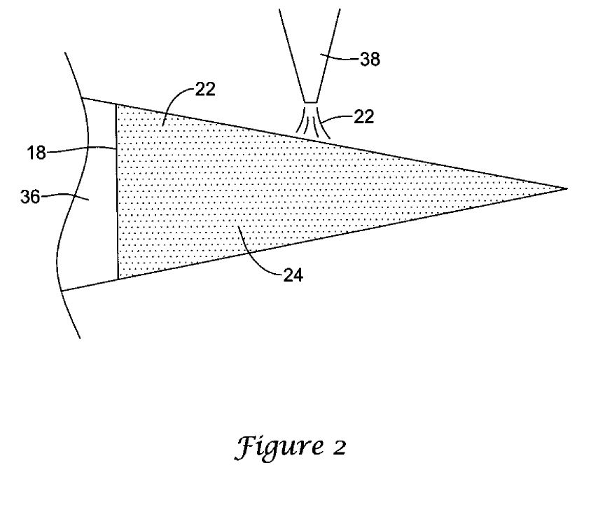

As indicated above, some of the steps contemplated for manufacturing filtering device 10 may include spray coating as depicted in FIG. 2. Spray coating may be desirable for a number of reasons. For example, spray coating may allow for filters to be manufactured that have a variety of different sizes and shapes. The manufacturing method may include providing a mandrel 36. Mandrel 36 may be generally similar to other mandrels used in the filter manufacturing art and may have a conical or tapered shape that is characteristic of typical filters. Filter loop 18 can be disposed on mandrel 36 and a spray coating apparatus 38 can be used to spray coat filter membrane 22 onto mandrel 36 and over filter loop 18.

In addition to what is described above, the manufacturing method may include any number of additional steps such as forming openings 24 in filter membrane 22. This may occur in any appropriate manner such as through the use of a laser or any other suitable cutting device. In addition, frame 18 may be attached to shaft 14. Numerous other manufacturing steps are also contemplated, as will be appreciated by those of ordinary skill in the art.

The materials that can be used for the various components of filtering device 10 (and/or the various components thereof) may include those commonly associated with filtering devices. For example, shaft 14, filter frame 18, and the like may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of filtering device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of filtering device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of filtering device 10 to achieve the same result.

In some embodiments, a degree of MRI compatibility is imparted into filtering device 10. For example, to enhance compatibility with Magnetic Resonance Imaging (MRI) machines, it may be desirable to make portions or all of filtering device 10 in a manner that would impart a degree of MRI compatibility. For example, portions or all of filtering device 10 may be made of a material that does not substantially distort the image and create substantial artifacts (artifacts are gaps in the image). Certain ferromagnetic materials, for example, may not be suitable because they may create artifacts in an MRI image. Alternatively, portions or all of filtering device 10 may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A coating, for example a lubricious, a hydrophilic, a protective, or other type of coating may be applied over portions or all of filtering device 10. Hydrophobic coatings such as fluoropolymers provide a dry lubricity which improves device handling and device exchanges. Lubricious coatings improve steerability and improve lesion crossing capability. Suitable hydrophilic and hydrophobic lubricious polymers are well known in the art and may include silicone and the like, polymers such as high-density polyethylene (HDPE), polytetrafluoroethylene (PTFE), polyarylene oxides, polyvinylpyrrolidones, polyvinylalcohols, polyethyleneoxides, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method for manufacturing a filter membrane for an embolic protection filter, the method comprising the steps of:
providing an elongate shaft having a proximal region and a distal region;
attaching a filter frame to the distal region;
attaching a filter membrane to the filter frame, the filter membrane having a plurality of apertures formed therein; and
wherein the filter membrane includes a polymer having a bulk portion and a surface portion, the surface portion including a surface modification.

2. The method of claim 1, wherein the bulk portion includes polyurethane.

3. The method of claim 1, wherein the surface portion is non-thrombogenic.

4. The method of claim 1, wherein the surface portion is lubricious.

5. The method of claim 1, wherein surface portion includes a polyethylene glycol.

6. The method of claim 1, wherein the surface portion includes a polypropylene oxide.

7. The method of claim 1, wherein the surface portion includes a polyethylene oxide.

8. The method of claim 1, wherein the surface portion includes a polypropylene oxide disposed between two blocks of a polyethylene oxide.

9. The method of claim 1, wherein the surface portion includes a polyacrylamide.

10. The method of claim 1, wherein the surface portion includes a polyvinylpyrrolidone.

11. The method of claim 1, wherein the step of attaching a filter membrane to the filter frame includes spray coating.

12. A method for manufacturing an embolic protection filter, the method comprising the steps of:
providing a filter frame;
providing a mandrel;
disposing the filter frame on the mandrel; and
spray coating a filter membrane onto the mandrel and the filter frame, wherein the filter membrane includes a polymer having a bulk portion and a surface portion, the surface portion including a surface modification.

13. The method of claim 12, further comprising the step of forming a plurality of apertures in the filter membrane.

14. An embolic protection filter, comprising:
an elongate shaft having a proximal region and a distal region;
a filter frame attached to the distal region; and
a filter membrane attached to the filter frame, wherein the filter membrane includes a perfluoropolyether urethane.

15. The filter of claim 14, wherein the surface portion imparts non-tackiness or non-blockiness.

16. The filter of claim 14, wherein the surface portion includes a low energy component, such as a perfluoro group.

17. The filter of claim 14, wherein the surface portion includes a low energy component, such as a poly(dimethyl siloxane) group.

18. The filter of claim 14, wherein the surface portion includes a low energy component, such as a hydrocarbon group.
